(19) United States
(12) Patent Application Publication
(10) Pub. No.: US 2021/0174928 A1
(43) Pub. Date: Jun. 10, 2021

(54) SYSTEM FOR CONTROLLING AN ENVIRONMENT TO ENHANCE CIRCADIAN RHYTHM

(71) Applicant: OK-TECHNOLOGIES, LLC, Ankeny, IA (US)

(72) Inventors: William R. Kahler, Ankeny, IA (US); Ted P. O'Brien, Ankeny, IA (US)

(21) Appl. No.: 16/705,475

(22) Filed: Dec. 6, 2019

(51) Int. Cl. G16H 20/30; G16H 50/30; G16H 10/60; G16H 40/67; G16H 15/00; G16H 50/70; G05B 13/02; F24F 11/30; H04L 29/08; A61M 21/02

(52) U.S. Cl. CPC G16H 20/30; G16H 50/30; G16H 10/60; G16H 40/67; G16H 15/00; F24F 2110/50; G05B 13/0265; F24F 11/30; H04L 67/12; A61M 21/02; G16H 50/70

(57) ABSTRACT

A system for controlling an environment to enhance circadian rhythm includes a control system associated with a building, and a plurality of sensors and environment enhancing devices placed within the building. Based on data transmitted by the sensors to the control system, the control system activates the environment enhancing devices to improve circadian rhythms of a resident.

### BACKGROUND OF THE INVENTION

This invention is directed to a system for enhancing an environment and more particularly for improving circadian rhythms.

Scientists have discovered that light is as essential to optimal health and development as nutrition and exercise. In nature, the sun delivers the entire spectrum of light needed by an individual's eyes and brain to align daytime with being awake and nighttime with preparing to sleep.

Traditional indoor lighting shines the same day and night, depriving the brain of a natural signal used to prepare body chemistry for sleep at night and maintain wakefulness during the day. Insufficient sunlight can make it harder for your brain to keep awake during the day. Conversely, light from LED bulbs, digital tablets and television screens can confuse the brain and prevent the natural process of preparing for sleep.

Circadian rhythm is a biological process that lasts twenty-four hours, and involves the physical, mental, and behavioral changes that follow a daily cycle. Circadian rhythms respond primarily to varying levels of light and darkness in our environment. The cycle regulates a number of important bodily functions such as body temperature, hormone production (testosterone, growth hormone, cortisol, and melatonin), cell regeneration, brain wave, activity, and most importantly, sleep. Other factors such as ambient temperature, stress, and exercise also can influence circadian rhythm, but none to the degree that light does.

Many people struggle with sleep, and in particular, people that suffer from brain ailments such as Alzheimer's, dementia, stroke, depression, or Parkinson's; all have difficulty getting a full night's sleep. The result is that those with brain ailments lose track of the normal day/night cycle, which adds to confusion, yelling, crying, and agitation that accompany these ailments. Additionally, because the body is not working in a normal twenty-four hour cycle, these people may forget to eat or are simply not hungry and don't drink enough fluids, which all add to ailments.

Accordingly, a system is needed that addresses these problems by improving the quality of life of both patients and caregivers.

### SUMMARY OF THE INVENTION

A system for controlling an environment to enhance circadian rhythm includes a control system associated with a building. The control system receives and transmits signals to and from a plurality of sensors and environment enhancing devices placed in the building.

The sensors detect movement, activity, temperature, and biometrics of a resident. The environment enhancing devices that mimic natural conditions related to circadian rhythm such as lights, thermostats, window treatments, aroma devices, sound devices and oxygen.

Based upon signals from the sensors, the control system controls the operation of the environment enhancing devices to create a healthy circadian rhythm for the resident based on the sensed information.

All data is collected, stored and analyzed to produce reports which permit the tweaking of the control system based upon the resident's results. Data is also collected and analyzed from multiple locations, and using mechanical learning programs and/or protocols are developed for the control system based upon the characteristics and condition of the resident.

### BRIEF DESCRIPTION OF THE DRAWINGS

FIG. 1 is a schematic of a system for controlling an environment; and

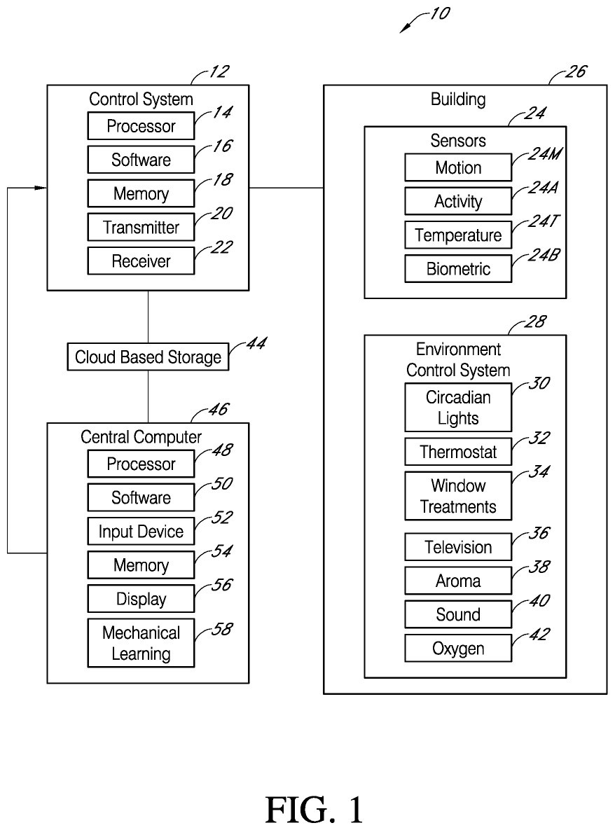

FIG. 2 is a schematic of a system for controlling an environment deployed in a building.

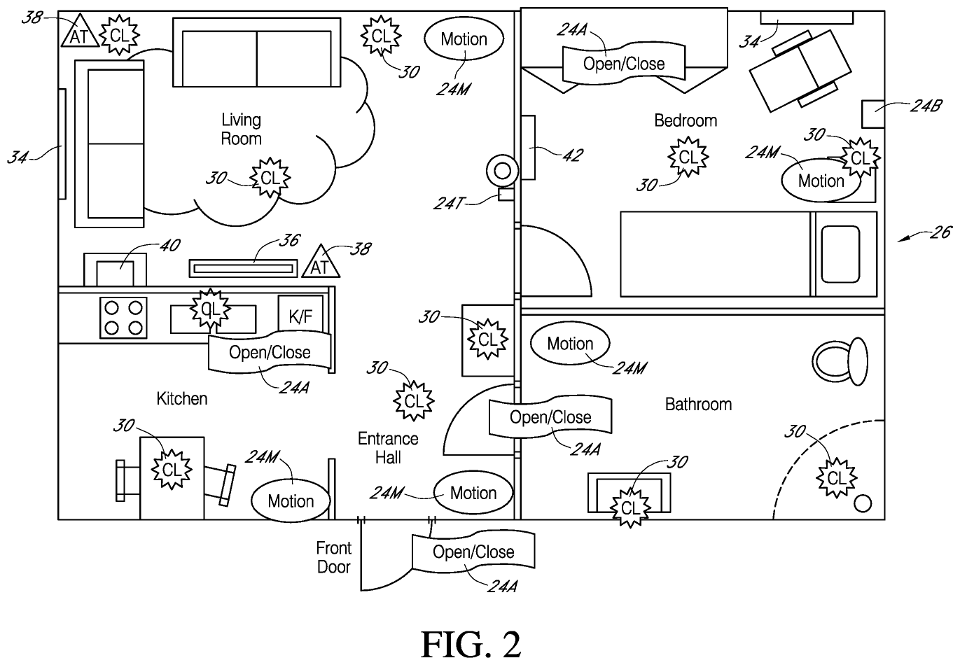

### DETAILED DESCRIPTION OF THE PREFERRED EMBODIMENTS

Referring to the Figures, a system 10 for controlling an environment to enhance circadian rhythm includes a control system 12 having a processor 14, software 16, memory 18, a transmitter 20, and a receiver 22. The control system 12 is connected to a plurality of sensors 24 that are placed within a building 26 such as a house, senior living environment or the like. The sensors 24 are of any type and are used to detect motion 24M, activity 24A, temperature 24T and/or biometrics 24B such as heart rate, breathing rate, blood pressure, and the like.

The control system 12 is also connected to a plurality of environment enhancing devices 28. The environment enhancing devices 28 are of many types and include circadian lights 30, thermostat 32, window treatments such as shades 34, television remote 36 to control the blue light level, aroma devices 38, sound devices 40, oxygen supply 42, door locks 43, and the like.

The control system 12 transmits and receives data to and from a cloud based storage 44. A central computer 46 is connected to the cloud based storage 44. The central computer 46 has a processor 48, software 50, input device 52, memory 54, display 56, and mechanical learning 58.

In operation, the sensors 24, environment enhancing devices 28, and control system 12, are placed within a building 26 preferably in primary areas of activity. As an example, activity sensors 24A are associated with the front door, bathroom door, refrigerator door, closet door, and the like, to sense the opening and closing of the doors, and track the coming and going of a resident and a caregiver. Motion detectors 24M are placed in all rooms to detect movement of the resident. A temperature sensor 24T is associated with the thermostat 32 and biomedical sensors 24B are placed preferably near the bed or alternatively other locations in the building.

Depending upon a purchased package and/or the condition/needs of the resident, environment enhancing devices 28 are placed throughout the different rooms. For example, circadian lights 30 are placed in lamps and light fixtures. Aroma therapy devices 38, sound devices 40, oxygen supply 42, shades 34, and controls for the television 36, are placed throughout the rooms. The control system 12 is placed in an out of the way place such as a closet or shelf.

The control system 12 is preset to operate the environmental enhancing devices 28 based on the resident's basic medical rating and desired living conditions. Once activated, the environment enhancing devices 28 are activated to allow artificial indoor conditions to mimic conditions that occur naturally in the environment. For example, the circadian lights 30, through the control system 12, are aware of the clock and automatically change the light spectrum to protect an individual's natural biological day and night without any loss of light intensity (lumens) or color quality. For example, during the day, blue and green colors are used by the brain to signal the body to be more active, while yellow and red signal the body to slow down.

Motion sensors 24M detect the movement of the resident and send a signal to the control system 12 which records the resident's movement. Based upon criteria, the control system 12 may activate environment enhancing devices 28 based upon motion sensor signals. For example, if a motion sensor 24M detects that a resident gets out of bed in the middle of the night, the control system would activate circadian lights 30 in a red spectrum to light a path to the bathroom and also light the bathroom. This can reduce falls as well as limit sleep interruption.

Data is also received from the activity sensors 24A, temperature sensor 24T, and biomedical sensors 24B by the control system 12. All data received is recorded and/or transmitted to the cloud based storage 44 where the data is stored for the individual resident.

The data predominantly is directed to a resident's activity including not only where they go, but how much time they spend in each area. As an example, the data might include information on time spent in each room, number of times in and out of bed, restroom use by time of day, door open and close count, time to bed at night, morning rise time, hours spent in bed during the day, average temperature, and the like.

The data is retrieved by the central computer and analyzed. In general the pattern of the resident as shown through the data is compared to an expected or more regular pattern. The analysis is provided to a caregiver, physician, and/or technician.

Preferably, the analysis is provided through weekly reports. From the reports a caregiver or physician will better understand what is happening in the residence when the caregiver is not there, provide an indication that the resident is on track, or alert caregivers, family, and physicians of advancements in a disease or other health issues that may arise.

From the analysis, the program can be tweaked or adjusted to improve the environment and living conditions and create a more regular pattern for the resident. For example, a physician or caregiver can provide instructions to a technician who makes changes to the software settings. These changes might include adjustments to the timing and intensity of lights, adjustments to temperature, refinements to the timing of aroma disbursement and the like. These changes are made by the technician and the new settings are downloaded to the control system 12 and activated.

Data is also combined by the central computer from multiple residences, and using machine learning, specific protocols are developed that are shown to work best based upon resident characteristics. Thus, if a resident has certain characteristics such as a specific basic medical rating, a specific program or protocol known to work best for the specific basic medical rating as determined from cumulative data, is transmitted to the control system to create the environment known to work best with a resident having those characteristics.

What is claimed is:

1. A system for controlling an environment to enhance circadian rhythm, comprising:
a control system associated with a building;
a plurality of sensors and environment enhancing devices placed in the building;
wherein the control system activates the environment enhancing devices based upon data received from the sensors about a resident of the building.

2. The system of claim 1 wherein the sensors include motion sensors, activity sensors, and a temperature sensor.

3. The system of claim 1 wherein the environment enhancing devices include circadian lights, a thermostat, and aroma devices.

4. The system of claim 1 wherein the control system transmits data received from the sensors to a cloud based storage.

5. The system of claim 4 wherein a central computer analyzes the stored data and adjusts the control system to enhance the environment of the building to improve circadian rhythms of the resident.

6. The system of claim 4 where a central computer analyzes data from multiple buildings and using mechanical learning adjusts the control system to enhance the environment of the building to improve the circadian rhythms of the resident based upon characteristics of the resident.
